(19) United States
(12) Patent Application Publication
(10) Pub. No.: US 2015/0211129 A1
(43) Pub. Date: Jul. 30, 2015

(54) Surface Treatment Process for Components Composed of Aluminium Having Detection of Impermissible Overheating

(71) Applicant: Premium Aerotech GmbH, Augsburg (DE)

(72) Inventors: Claudia Juricic, Muenchen (DE); Melanie Markgraf, Augsburg (DE)

(21) Appl. No.: 14/420,158

(22) PCT Filed: Aug. 7, 2013
(86) PCT No.: PCT/DE2013/000442

§ 371 Date: Feb. 6, 2015

(30) Foreign Application Priority Data
Aug. 8, 2012 (DE) 10 2012 015 579.4

(51) Int. Cl. C23G 1/00; G01N 21/88; G01N 33/20; C23G 1/12; C23G 1/22

(52) U.S. Cl. CPC C23G 1/00; C23G 1/125; C23G 1/22; G01N 33/20; G01N 21/8803

(57) ABSTRACT

A surface treatment method for components made of aluminum or aluminum alloys, involving treatment steps of alkaline cleaning, rinsing, alkaline pickling, rinsing, acid pickling, rinsing, also involves a step of conducting a visual inspection for soft spots after the alkaline cleaning in step or the alkaline pickling.

### BACKGROUND AND SUMMARY OF THE INVENTION

Exemplary embodiments of the invention relate to a surface treatment method, in particular to a surface protection method or a surface coating method, including an inspection for inadmissible local overheating for components made of aluminum or aluminum alloys, which contains a pre-process including alkaline cleaning and/or alkaline pickling as a sub-process step. It further relates to a method for testing such components for soft spots in a surface treatment method.

The term “soft spot,” also called “weak spot,” “hot spot” or “partial material affection (PMA),” refers to inadmissible local overheating, which can occur during machining, in particular with high-strength aluminum alloys. Overheating may be the result of unfavorably selected machining parameters, tool failure or when reduced heat dissipation takes place. It leads to a local drop in hardness of the component and mechanical strength, and in a local change of electrical and thermal conductivity. “Soft spots” can be detected either by measuring the hardness or electrical conductivity or by thermographic analyses. However, given the significant complexity that is required to do so, only a visual inspection is frequently carried out in practical applications, since “soft spots” appear as pronounced dark spots, for example after chromic acid anodizing (CAA). The dark spots are caused by the formation of a thicker anodization layer in the overheated surface region of the component.

However, a comparable visual inspection for soft spots, for example, using the more environmentally friendly method according to the principle of tartaric sulfuric anodizing (TSA), is no longer possible after the anodizing process has been concluded. This is because TSA, in contrast to CAA, generates a transparent anodization layer, in which the formation of a thicker layer cannot be detected with the naked eye. As a result, no testing method that is suitable for series testing machined components for weak spots is available at present.

Accordingly, exemplary embodiments of the invention are directed to a visual inspection technique for soft spots can be carried out in almost all surface treatment methods for aluminum and aluminum alloys which include alkaline cleaning and/or alkaline pickling.

According to exemplary embodiments of the invention a separate step is provided, which involves performing the visual inspection for soft spots in a surface treatment method for components made of aluminum or aluminum alloys, in which the components are subjected to alkaline cleaning and/or alkaline pickling treatment steps in a pre-process, this separate step following alkaline cleaning or alkaline pickling. If there are no apparent soft spots during the visual inspection after rinsing following the alkaline cleaning step, a further visual inspection for soft spots can also be carried out after rinsing following the alkaline pickling step. The step of the visual inspection can thus also take place after rinsing following alkaline cleaning and after rinsing following alkaline pickling.

Surface treatment methods shall be understood to mean, among other things, surface protection or surface coating methods, such as chromic acid anodizing (CAA), sulfuric acid anodizing (SAA), tartaric sulfuric anodizing (TSA), phosphoric sulfuric anodizing (PSA), hard sulfuric anodizing (HSA), or chemical conversion coating (CCC).

A pre-process is used in particular to clean the aluminum components, so as to subsequently be able to carry out a successful, which is to say in particular defect-free, surface protection or surface coating method. It typically comprises the steps of alkaline cleaning, alkaline pickling and acid pickling, wherein single or multiple intermediate rinsing steps (hereinafter referred to as rinsing for the sake of simplicity) are carried in each case between the steps. Alkaline cleaners and pickle liquors contain chemical substances that form an alkaline solution, such as NaOH, KOH, Ca(OH)₂. Suitable cleaners are sold by the designation P3-Almeco 18 by HENKEL, or by the designation TURCO 4215 NC by TURCO CHEMIE GmbH, for example. Suitable pickle liquors are available by the designation ALUMINETCH from HENKEL SURFACE TREATMENTS, for example. Further examples of alkaline pickle liquors can be found in U.S. Pat. No. 4,383,042.

The pre-processes additionally include a penetrant test, which many components must undergo prior to a surface protection process. Since the components are wetted with a penetrant during this test, they pass through another pre-process after the penetrant test has been completed, so as to be available subsequently in cleaned form for a surface protection process.

The invention consequently turns away from carrying out a visual inspection for soft spots only after a surface treatment process has been completed, for example, only after a complete surface protection method according to the principle of anodizing, or only after a penetrant test of a component. Instead the invention pursues the principle of interrupting the pre-process of the surface treatment method in question during or after the process step of alkaline cleaning and/or alkaline pickling, so as to carry out a visual inspection of the components for soft spots. This is because it was recognized as a result of the invention that the “soft spots” already appear after alkaline cleaning and/or after alkaline pickling in locations where previously inadmissible overheating has occurred. Depending on the viewing direction and incidence of light, they appear after alkaline cleaning in a milky-white to brownish color in an otherwise metal surface. After alkaline pickling, they become visible as slightly brownish spots, optionally in a compact black layer, which is created during alkaline pickling due to copper being cemented out of the copper-containing aluminum alloy on the remaining component surface. Subsequent acid pickling removes the black layer, along with the brownish spots, so that a deviation in the material strength and structural conditions is no longer visually no discernible. According to the invention, a visual inspection is thus carried out at the earliest after alkaline cleaning, and at the latest prior to conventional acid pickling or the complete removal of the substantially black layer.

Since the inspection for soft spots can now be carried out as part of the pre-process of a surface treatment process, the method according to the invention is limited not only to a certain surface protection method, as was previously the case with CAA, during which the soft spots were not detected until after the component had passed through the entire process. Instead it can be applied to all pre-processes and surface protection processes in which alkaline cleaning and/or alkaline pickling represent a sub-process. Since, according to the invention, the visual inspection for soft spots takes place in the pre-process, the component no longer has to pass through the entire surface protection process in vain if the findings are positive, which is to say if soft spots are discovered, but can be sorted out at an earlier stage. This saves time and material, whereby the surface treatment method becomes less expensive. Many components must additionally undergo a penetrant test prior to a surface protection process. The visual inspection for soft spots according to the invention can now also be carried out prior to the complex penetrant test. If the findings are positive, the penetrant test can thus be saved, which otherwise would be carried out unnecessarily. The cost-effectiveness of the surface treatment process can thus also be increased.

The color changes on the component are consistent with experimentally conducted measurements with respect to the electrical conductivity of soft spots detected according to the invention. For example, the electrical conductivity of the material is reduced within a circular brown spot. In the case of annular overheating, higher electrical conductivity can be found at the center than in the annular region. However, the drop in electrical conductivity in the most unfavorable case (lithium-containing aluminum alloys) is only approximately 8% and thus very complex to detect by way of measurement. Measuring electrical conductivity has therefore not been an option for series testing thus far.

The soft spots are not detected based on objective or objectifiable measurement results, but during the course of a visual inspection. In some aluminum alloys, the color difference between the black layer and the brown spots is sufficiently clearly apparent. The result of the visual inspection can be improved by carrying out an additional step of drying the component, using methods that are known per se, prior to the step of visual inspection according to one advantageous embodiment of the invention. In the dry state of the components, the color differences between the brown spots on the one hand, and the black layer on the other hand, are in many cases more clearly apparent. After completion of the visual inspection, the dried component can be directly subjected to the method step of acid pickling. Advantageously, however, it is supplied to an entire pre-process again, so as to continue to be treated “wet in wet” at the latest with the method step of acid pickling.

According to a further advantageous embodiment of the invention, drying of the components can be carried out by way of compressed air in the additional step. This achieves a faster drying result, which moreover can be directed more effectively at the component surface to be dried. This allows the energy expenditure for drying to be reduced.

In particular, in the case of only minor local overheating, mere drying of the components does not produce a sufficiently reliably discernible color difference between the brown spots and the black layer. According to a further advantageous embodiment of the invention, the black layer can thus be removed as an alternative or in addition, without eliminating the brown spots. The visually discernible difference between the brown spots on the one hand, and the black layer on the other hand, is based on a different layer thickness. Due to differing refraction, it evokes a deviating color impression between the black layer and the brown spots. The latter are created by a locally higher layer thickness. By evenly ablating a layer of uniform thickness, the black layer can be completely ablated, while the soft spots remain marked in color due to the higher layer thickness above them. This results in an even more clearly perceptible color difference between the intact regions of a component and the soft spots thereof. A sufficient, which is to say visually perceptible, color difference between the black layer and the brown spots can optionally already be achieved by not removing the black layer completely, but only partially. Depending on the technology that is used for ablation, prior or subsequent drying can be eliminated, and the method can optionally be carried out “wet in wet.”

The removal or ablation of the black layer, or of a portion of the black layer, in an even and uniform thickness can take place, for example, by way of mechanical methods, such as compressed air, water jet, sandblasting, sound or vibration methods, or by way of mechanical brushing. All these methods have in common that they can be employed in a planar manner and allow the black layer, or a portion thereof, to be evenly ablated in a planar manner. According to an advantageous embodiment of this method, the ablation of (a portion of) the black layer can take place already during the prior rinsing step, subsequent to alkaline pickling. For this purpose, the rinsing liquid or the water can be directed at the component to be rinsed in a water jet nozzle bath having many small nozzles. The rinsing and ablation of the black layer can consequently be combined in one pressure rinsing step, whereby the procedure of the method according to the invention can be simplified, which is to say shortened and made less expensive.

According to a further advantageous embodiment of the invention, the complete or partial removal of the black layer can alternatively or additionally be carried out by way of chemical means. These can likewise be applied in an evenly distributed planar manner, so that the thickness of the black layer can be evenly reduced or the same can be completely ablated, without the remaining layer on the soft spots being removed at the same time. A selection of the mechanical or chemical method, or a combination thereof, can be ascertained based on laboratory experiments and selected for large-scale use.

According to a further advantageous embodiment of the invention, the black layer can be removed using a modified acid pickle liquor, as it is already used in principle in the pre-process after the alkaline pickling step. However, deviating from this, the physical-chemical action of the modified acid pickle liquor on the component is reduced so much that it removes at most the black layer, not however the brown spots. The modification of the additional acid pickle liquor according to the invention can be caused by a lower concentration, by a shorter application time, or by a reduced temperature of the acid pickle liquor, under otherwise unchanged conditions. A combination of these parameters, for example a lower concentration together with a shorter application time, optionally as an alternative or in addition to a lower temperature of the acid pickle liquor, is also possible. As an alternative, one of the parameters can be increased in favor of the reduction of one or more other parameters. For example, the application time and/or the temperature of an acid pickle liquor having a drastically reduced concentration can be considerably extended or increased. A short application time and/or a high temperature in conjunction with an increased concentration of the acid pickle liquor is also possible to preserve the soft spots, while the black layer is removed. In addition or as an alternative, the pickling bath can be moved so as to increase the action of a reduced parameter.

Since the black layer would have been removed anyways during the regular step of acid pickling, a portion of this step can already be anticipated in the inventive method, so as to be able to carry out the visual inspection for soft spots. The subsequent, regular step of acid pickling thus only needs to be carried out to a lesser degree, whereby material, time and energy can be saved.

In the three above-mentioned methods, a black layer is formed, which is ablated at least partially so as to have the soft spots emerge more clearly. It is created by the step of alkaline pickling. According to a further advantageous embodiment of the invention, pickling using a modified alkaline pickle liquor can be carried out in a subsequent step after a conventional “alkaline cleaning” step and the subsequent “rinsing” step, wherein this can be followed by the visual inspection and the known steps of “alkaline pickling” and “rinsing.” A conventional alkaline pickle liquor, in particular having a normal concentration, is used for the alkaline pickling step after the visual inspection.

The purpose of the modification of the additional alkaline pickle liquor according to the invention is to expose the component to decreased or reduced physical-chemical action of the alkaline pickle liquor, so as to only highlight the soft spots, without forming the black layer. The reduced action can additionally be caused by a lower concentration, by a shorter application time, or by a reduced temperature of the alkaline pickle liquor, under otherwise unchanged conditions. A combination of these parameters, for example a lower concentration together with a shorter application time, optionally as an alternative or in addition to a lower temperature of the alkaline pickle liquor, is also possible. As an alternative, one of the parameters can be increased in favor of the reduction of one or more other parameters. For example, the application time and/or the temperature of an alkaline pickle liquor having a drastically reduced concentration can be considerably extended or increased. A short application time and/or a high temperature in conjunction with an increased concentration of the alkaline pickle liquor is also a way to accentuate the soft spots, before the black layer is created. In addition or as an alternative, the alkaline pickling bath can be moved so as to increase the action of a reduced parameter.

A suitable concentration of the modified pickle liquor ranges between 9 g and 18 g of pickle liquor for 1 liter of distilled water, for example, compared to a concentration of a conventional pickling bath of 38 g/l. The temperature thereof can range between a room temperature of approximately 20° C. and an elevated temperature of 42° C. Time periods from 5 to 240 seconds, for example, can be selected as immersion times.

In addition to, or even prior to, regular alkaline pickling, an additional alkaline pickling step in a pickling bath having a reduced physical-chemical action is thus integrated into the method, so that only the soft spots become apparent when components containing soft spots are immersed, without the compact black layer being formed. After the component has been immersed into the additional pickling bath, it only needs to be rinsed, so as to then be subjected directly to a visual inspection. This method thus also takes place “wet in wet.” The removal of the black layer can consequently be eliminated, which simplifies the procedure of the method according to the invention.

If the visual inspection supplied positive findings, which is to say the discovery of soft spots, initially only a component deviation can be established, without immediately categorizing the component as scrap. The establishment of a component deviation can be followed by a standardized technical inspection process, during which it is analyzed, according to defined criteria, and decided whether the component can nonetheless be used (“used as-is”), whether it can be repaired, or whether it is categorized as scrap. If this process takes longer, for example one or more days, and the component, which can be used further or repaired, cannot be kept “wet” during this time, according to a further advantageous embodiment of the method according to the invention it can pass through the entire, normal pre-process once again, except this time uninterrupted. It can thus be ensured that even a component which resulted in positive findings during the visual inspection, but can nonetheless still be used, satisfies the same quality criteria as a component without findings.

The alkaline cleaning, the alkaline pickling and the acid pickling, as well as the subsequent anodizing steps, can be carried out in a largely mechanized, and optionally automated, manner. According to a further advantageous embodiment of the invention, the visual inspection for soft spots can also be carried out automatically. For this purpose, the components to be inspected can be visually captured by way of a camera, and the recorded images can be analyzed for color differences by way of image detection software and evaluated. The visual inspection can thus be objectified and verifiably documented, whereby it can be easily integrated not only in the procedure of the anodizing method, but also in a quality assurance process. Moreover, the deployment of an employee for the visual inspection in an environment of, in some instances, aggressive acids and lyes can be dispensed with.

According to a further advantageous embodiment of the method according the invention, a penetrant test and/or a surface protection method according to one of the above-mentioned principles can be carried out after the last rinsing of the pre-process. Since the component is wetted with a penetrant during the penetrant test, which impedes a surface protection method, it can be subjected to another complete pre-process after the penetrant test has been successfully completed. Without such a penetrant test, it can be supplied directly to a surface protection method, without the risk of undergoing unnecessary treatment due to soft spots and being rejected later.

In the method for testing components made of aluminum or aluminum alloys for soft spots in a surface treatment method, which contains a pre-process including alkaline pickling and/or acid pickling as a sub-process, a visual inspection for soft spots is performed prior to the alkaline pickling step and/or prior to the acid pickling step. This method exhibits the advantages already described above and can be varied in the manner described above.

### BRIEF DESCRIPTION OF THE DRAWING FIGURES

The principle of the invention will be described in more detail hereafter based on drawings. In the drawings:

FIG. 1 shows a pre-process according to the prior art;

FIG. 2 shows the method according to the invention in the most general form thereof;

FIG. 3 shows one embodiment for special aluminum alloys;

FIG. 4 shows a variant including a visual inspection prior to alkaline pickling;

FIG. 5 shows a variant including a visual inspection prior to acid pickling;

FIG. 6 shows one embodiment of the preceding variant including a prior drying step; and

FIG. 7 shows a specific embodiment of the method according to FIG. 6.

### DETAILED DESCRIPTION

A conventional pre-process for a surface protection method for components made of aluminum or aluminum alloys, which is known per se, starts with degreasing and pickling baths, which include alkaline and acid pickling steps and are used to clean the component. Intermediate rinsing is carried out between the cleaning and pickling baths, so as not to contaminate the subsequent bath with the chemicals of the baths. For this purpose, the component is, or multiple components are, suspended from a rack so as to be treated in dipping baths. The processing steps can thus advantageously be automated so the operator has as little as possible in contact with the chemicals of the dipping baths. For improved efficiency of the cleaning and pickling baths, the components or the baths can be moved, the latter by way of stirring, injecting compressed air or vibrating the container, for example.

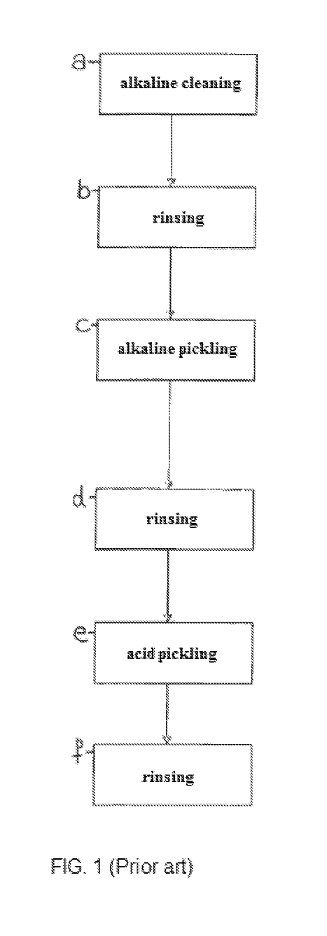

According to FIG. 1, “alkaline cleaning” of the components in a first step a) is followed by “rinsing” in a step b), so as not to contaminate the following steps with liquid residue from the cleaning step a). Thereafter, “alkaline pickling” follows in a step c). This is where the grayish-brown to predominantly black layer on the treated component is created. In a further step d), the components are rinsed and freed of the residue of the alkaline pickle liquor. In a subsequent step e), they are subjected to “acid pickling.” In a step f) following thereafter, the components are rinsed in the known manner, so as to then be subjected to a penetrant test or the surface protection method.

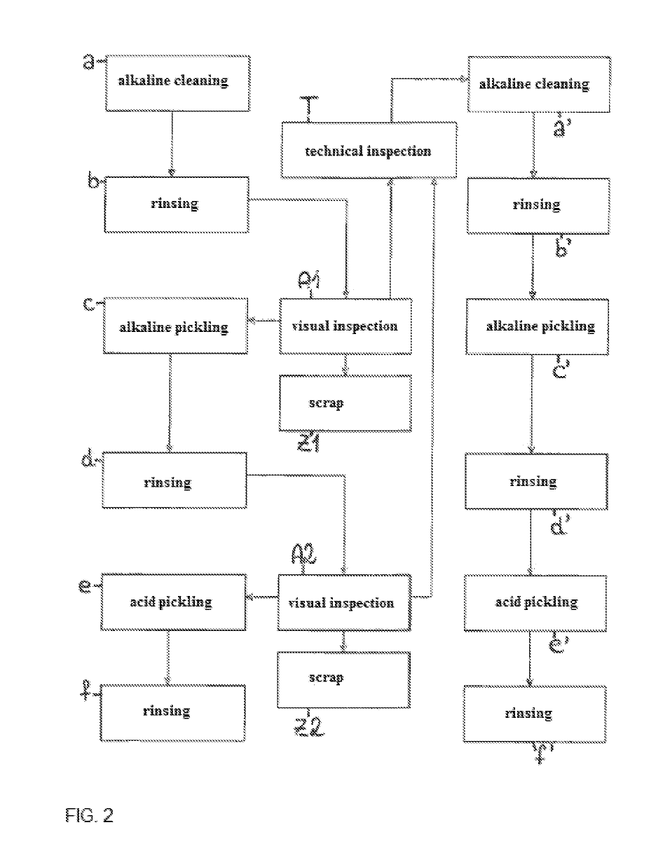

FIG. 2 shows the principle of the invention in a relatively general representation. It illustrates that the method is based on a pre-process comprising steps a) through f) according to the prior art, which is represented largely unchanged on the left-hand side or as the left-hand column. The principle of the invention is based on the pre-process being interrupted after the “alkaline cleaning” in step a), or after the “rinsing” in step b) following thereafter, and/or after the “alkaline pickling” in step c), or after the subsequent step d), the “rinsing” step”, so as to carry out a visual inspection A1 and/or A2. Thus, in principle, method steps a) through f) are carried out unchanged in all method variants of the invention, wherein additional steps are inserted between steps b) and c) and/or between steps d) and e) in connection with a visual inspection A, or A1 and/or A2.

In a first aspect, the invention is based on the realization that milky-white to slightly brown spots form in locations in which inadmissible local overheating has taken place during a preceding machining step of the component. Based on the brown spots, the overheated locations, which result in locally delimited reduced hardness of the component, known as “soft spots,” can be visually identified. The soft spots are already apparent after the “alkaline cleaning” in step a) or after the subsequent “rinsing” in step b). The pre-process can thus already be interrupted after step a), or after step b), so to examine the component for soft spots. The sooner a damaged component can be identified and separated from the processing process, the less unnecessary costs it causes. The early point in time of detecting potential soft spots thus contributes to a cost reduction of the processing process.

If the visual inspection A1 according to FIG. 2 produces undoubtedly positive findings, which is to say unacceptable soft spots of the examined component, the component is sorted out as scrap Z1. In cases of doubt, however, the component can be subjected, after the visual inspection A1, to a standardized technical inspection process T during which it is examined whether the detected deviation in the specific instance can be tolerated (also referred to as “used as-is”), whether it can be repaired, or whether it is indeed irreparable and thus only fit for scrap. The technical inspection process can take longer, for example one or more days, so that the component cannot be kept wet during this time. This process causes the component not to be able to be supplied unchanged to the ongoing pre-process, which is to say to the subsequent step c) of “alkaline pickling.” Rather, the component must again be subjected to step a′) of “alkaline cleaning step b′) of “rinsing.” For the sake of clarity, a complete pre-process is shown on the right-hand side or in the right-hand column of FIG. 2; however, it is composed of the same steps a) through f) as the left-hand column of FIG. 2. De facto, the “alkaline cleaning” of step a) is thus repeated as step a′), and rinsing according to step b) or b′) is carried out, whereupon the interrupted pre-process of the left-hand column is continued in step c′) with “alkaline pickling.”

If no positive findings should emerge from the visual inspection A1, the pre-process is continued with known step c) of “alkaline pickling.” This is because positive findings will emerge sooner in the visual inspection A1, the more pronounced the damaging heat input into the component was. If overheating was less intensive, it may not show until perhaps a later method step. To be on the safe side, a further visual inspection A2 is thus carried out after the subsequent step d) of “rinsing.”

This is because a grayish-brown to predominantly black layer (hereafter referred to as the “black layer” for the sake of simplicity) is created on the treated component during “alkaline pickling” in step c). It is owing to the invention to have explored as a further aspect that slightly brownish spots form in the black layer on “soft spots” of the component. Based on the brown spots, overheated locations, which result in locally delimited reduced hardness of the component, can be visually identified. However, the acid pickling in step e) would eradicate this surface effect again. Additionally or exclusively after “alkaline pickling” in step c), or the subsequent “rinsing” in step d), the component can thus be subjected to a visual inspection A2 so as to examine it for soft spots. After the visual inspection in A2, just like after the inspection in step A1, the component can either immediately be categorized as scrap Z2 or be subjected to the technical inspection T for a component deviation. The further treatment thereof after the technical inspection T thus corresponds to that described above insofar as again the “alkaline cleaning” as step a′) and the “rinsing” as step b′) are repeated, however now additionally the “alkaline pickling” as step c′), the “rinsing” as step d′), and the “acid pickling” as step e′) are also added, so as to end the pre-process with the “rinsing” of step f) or f′).

Should the component not show any positive findings in the visual inspection A2, the pre-process is ended after the “acid pickling” in step e) and after the subsequent “rinsing” in step f).

Thereafter, it is supplied to a penetrant test and/or the planned surface protection method.

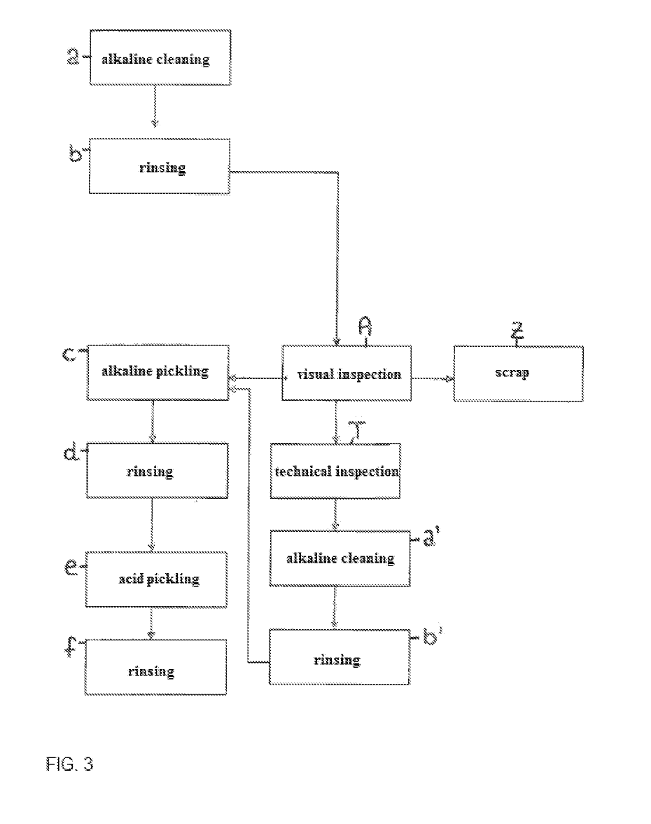

FIG. 3 describes a simplified method for the aluminum alloy AA2196. According to the invention, it was possible to establish that this alloy shows all soft spots already after “alkaline cleaning” in step a) and the associated “rinsing” in step b). The visibility of the soft spots is thus dependent on the degree of the previously introduced overheating into the component, the heat sensitivity of the material, which is to say the temperature stability thereof, and the alloying constituents. Thus, the visual inspection A after “rinsing” in step b) already reliably shows findings on a damaged component. As a result, the component can either be categorized as scrap Z or be supplied to a technical inspection T, and thus again to “alkaline cleaning” in step a′) and the associated “rinsing” in step b′) or, without findings, it can be supplied directly to “alkaline pickling” in step c) and the further pre-process comprising steps d) through f). In any case, for this alloy this results in a simplified, and thus even more cost-effective, test process for soft spots.

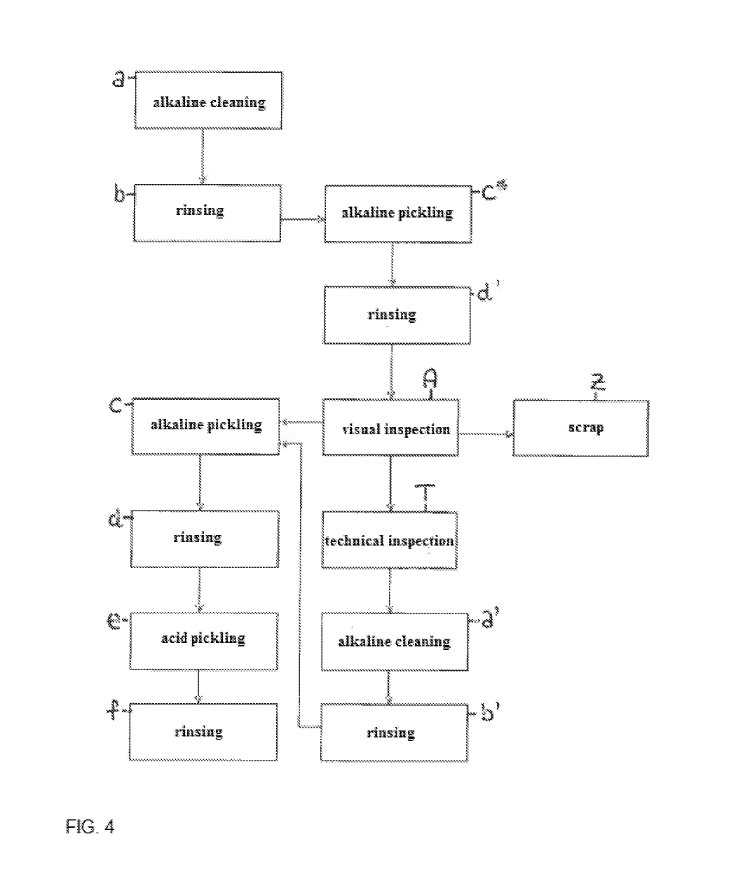

As previously mentioned, alkaline pickling of the components results in a grayish-brown to predominantly black layer, from which the soft spots can set themselves apart by slightly lighter, light brown spots. Depending on the pickle liquors used, the concentration thereof, the temperature of the pickling bath, the application time, and the alloy of the component, the soft spots can be apparent more or less clearly. FIG. 4 thus shows a variant of the inventive method, which after “alkaline cleaning” in a) and the associated “rinsing” in step b), and prior to the visual inspection A, provides for another step c*) for “alkaline pickling” and an associated step d′) for “rinsing.” According to the invention, in principle the same pickle liquor is used for the “alkaline pickling” of step c*) as for the “alkaline pickling” in step c) of the pre-process, at a substantially identical application time and identical temperature, however at a reduced concentration. The decisive factor is that the “alkaline pickling” in step c*) subjects the aluminum component to lower physical-chemical action, and thus renders the soft spots, or the brownish spots indicating the same, visible without the black layer being generated. In this way, a more reliable result can be achieved in the subsequent visual inspection A, so that the inspected component, as before, can either be supplied to the interrupted pre-process at step c) with the “alkaline cleaning”, and the subsequent steps d) through f) if no findings are made, or continues with renewed alkaline cleaning in step a′), an associated “rinsing” in step b′), and subsequently continues the pre-process with step c), or ends up in scrap Z, in the event of findings after a technical inspection T.

Suitable parameters for formulations that vary compared to this and have different concentrations, temperatures and application times of the alkaline pickle liquor according to step c*) can be found in the following table:

TABLE 1
Exemplary formulations for step c*)

| Concentration | Temperature | Residence time |
|---|---|---|
| 36 g/l | 42° C. +/− 2° C. | 20 sec. |
| 18 g/l | 20° C. +/− 2° C. | 90 sec. |
| 9 g/l | 20° C. +/− 2° C. | 180 sec. |

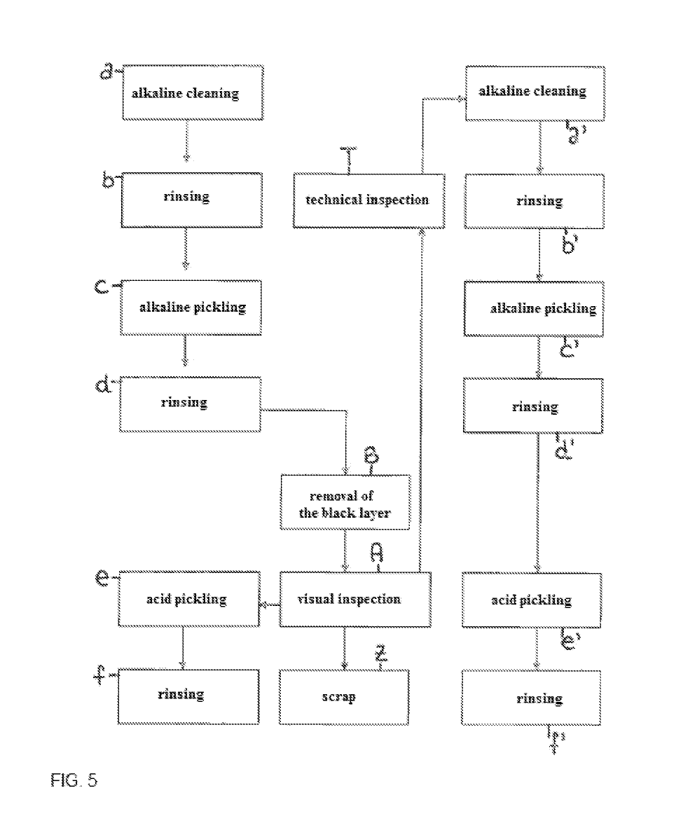

FIG. 5 shows an alternative method, where the interruption of the pre-process is not carried out prior to the “alkaline pickling” in step c), but prior to the “acid pickling” in step e). As mentioned before, the “alkaline pickling” in step c) results in the black layer on the treated component, in which the damaged overhead locations can become apparent as slightly brownish spots. Unlike the previous method, the method according to the invention in accordance with FIG. 5 ensures increased visibility of the brown spots by removing the black layer at least partially in a step B after the “rinsing” in step d) and prior to the visual inspection A. The special characteristic of this method step is that the intensity of the machining process is only so much that merely the black layer is removed, not however the brown spots. Compressed air can be used for this purpose. However, water jets, sandblasting, mechanical machining method such as brushing, or the use of vibration or sound, or special cleaning agents, are also conceivable to remove the black layer. Depending on the selection of the specific method for removing the black layer, it can be combined with the preceding method step d) for rinsing the components or make the same unnecessary. For example, nozzles in the rinsing bath in step d) can direct a plurality of water jets under pressure at the component, which at least partially ablate the black layer.

The decisive factor in any case is that the applied method in method step B ablates only the black layer, or optionally also only a portion thereof, so much that a clear color difference between the layer, or the areas of the component from which the layer has been removed, and the brown spots is apparent. For this purpose, a layer measuring a few micrometers is to be ablated, such as in the range of 0.1 to 10 μm.

After at least a portion of the black layer has been removed, the component is prepared for a visual inspection in the subsequent step A. Now, the brown spots, and thus the soft spots of the material of the components, can be identified and localized, and the components are marked and sorted immediately or later. In addition to a visual inspection of the components by trained staff, the step can also be carried out with the aid of cameras and thus be automated.

After the visual inspection has been completed, in any case the components that have not been sorted are further processed in the known manner, which is to say subjected to an acid pickling process in a subsequent step e). In a step f) following thereafter, the components are rinsed in the known manner, so as to then be subjected to a penetrant test or the surface protection method.

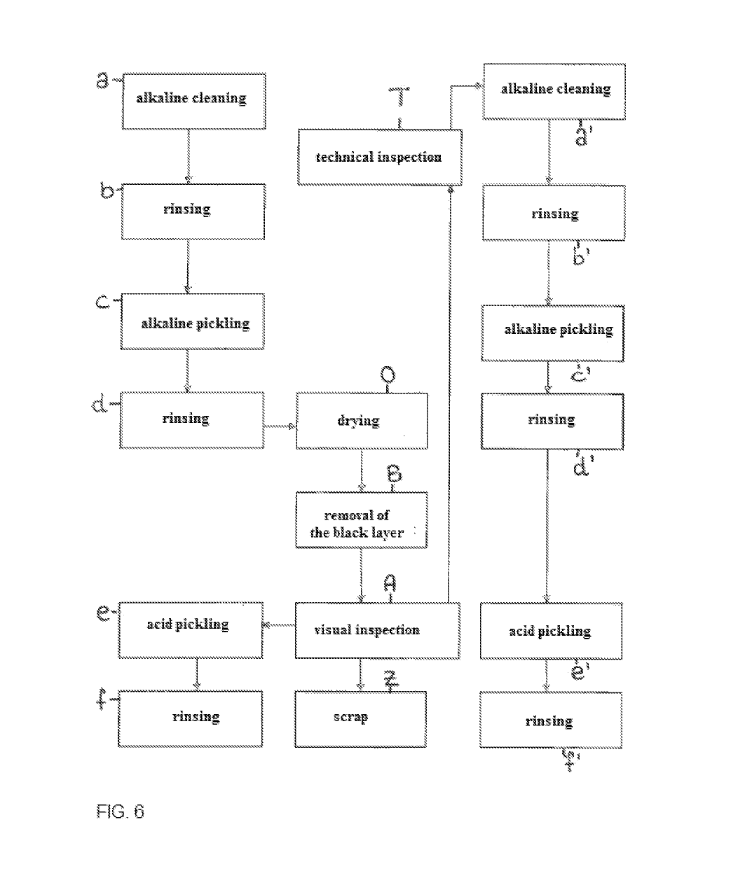

FIG. 6 shows an alternative to the preceding method according to FIG. 5, which takes advantage of a further realization for highlighting the brownish spots as compared to the black layer: in some aluminum alloys, sufficient color differences are already apparent after the “alkaline pickling” in c) and the subsequent “rinsing” in step d) if the component is dried. According to the invention, the pre-process is thus interrupted after step d): In a step O, the components are first dried. This step is used to free the components of moisture from the rinsing process in step d). The drying can take place in the known manner by storage in warm air and supported by the use oil-free and anhydrous compressed air. Rinsing with warm water in the preceding step d) can also lead to a faster drying result. Depending on the selection of the specific method for removing the black layer in step B, it can be combined with the preceding method step O for drying the components or make the same unnecessary. For example, when compressed air is used to remove the black layer, it also causes the components to dry, so that steps O and B coincide.

The known method steps follow the visual inspection A, subsequent to which a component without findings is supplied to the pre-process, where it is supplied to the “acid pickling” in step e) and the subsequent “rinsing” in step f). A component having clear findings can be categorized as scrap Z or assigned to the technical inspection Z for a component deviation, which requires the initial steps a) through d) of the pre-process to be repeated.

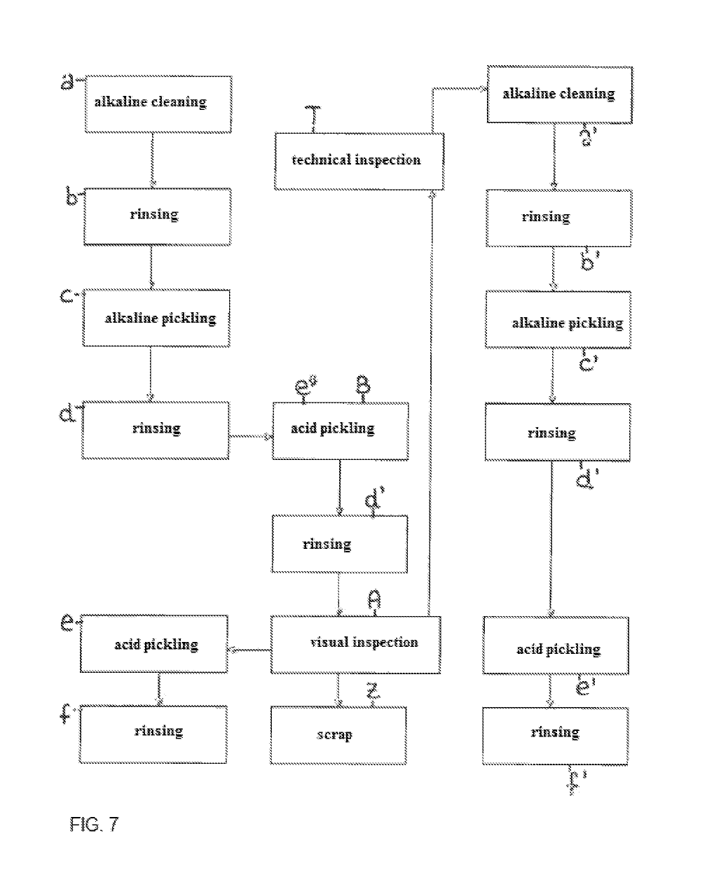

FIG. 7 shows a further variant of the method according to FIG. 5. It takes advantage of the realization that the “acid pickling” in step e) eliminates the black layer. According to the invention, the method is thus interrupted after “rinsing” in d), and prior to the visual inspection A, with a step e*) in which “acid pickling” is carried out at a reduced concentration. This method step utilizes the same pickle liquor as in the pre-process of step e), however due to the reduced concentration results in only a partial ablation of the black layer, leaving the brownish spots substantially untouched. Step e*) consequently replaces step B according to FIG. 4 by at least partially removing the black layer. After a subsequent step d′), in which the components are rinsed, these can be supplied to the visual inspection A. The remaining method corresponds to that of FIG. 5.

The above methods described in detail are exemplary embodiments and they can be modified by a person skilled in the art in the customary manner within a broad scope without departing the scope of the invention. In particular, the listed specific formulations of the cleaning and pickling paths can be composed in a different manner than that described herein, and above all be varied in terms of the concentrations, temperatures and residence times. Likewise, the procedures such as the immersion of the components can take place in a different manner, for example by way of spraying or wiping, or the rinsing step can be carried out not only once, but multiple times, for example when this lends itself for procedural reasons. Finally, the use of the indefinite articles “a” or “an” does not preclude the features in question from being present multiple times or several times.

### LIST OF REFERENCE NUMERALS

a), a′) alkaline cleaning
b), b′) rinsing
c), c′) alkaline pickling
d), d′) rinsing
e), e′) acid pickling
f, f′) rinsing
c*) alkaline pickling at a reduced concentration
e*) acid pickling at a reduced concentration
A, A1, A2 visual inspection
B removal of the oxide layer
O drying
T technical inspection
Z, Z1, Z2 scrap

1-13. (canceled)

14. A surface treatment method for components made of aluminum or aluminum alloys, comprising a pre-process including the following treatment steps:
a) alkaline cleaning;
b) rinsing;
c) alkaline pickling;
d) rinsing;
e) acid pickling;
f) rinsing;
wherein a visual inspection for soft spots on the components is performed after the rinsing in step b) or after the rinsing in step d).

15. The method of claim 14, further comprising:
an additional step of drying the component, which is performed prior to the visual inspection.

16. The method of claim 15, wherein the additional step of drying is performed using compressed air.

17. The method of claim 14, further comprising:
removing a black layer on the components in a separate step after the rinsing in step d), and prior to the visual inspection.

18. The method of claim 17, wherein the black layer is mechanically or chemically removed.

19. The method of claim 18, wherein the chemical removal involves using an acid pickle liquor with a reduced concentration, a reduced application time, or a reduced temperature.

20. The method of claim 14, further comprising:
performing alkaline pickling using a pickle liquor having a lower concentration, a reduced application time, or a reduced temperature in an additional step after the alkaline cleaning in step a) and prior to the visual inspection.

21. The method of claim 14, wherein the steps a) of alkaline cleaning and b) of rinsing are performed between the visual inspection and the alkaline pickling in step c).

22. The method of claim 14, wherein the steps of a) alkaline cleaning, b) rinsing, c) alkaline pickling, and d) rinsing are performed between the visual inspection and the acid pickling in step e).

23. The method of claim 14, further comprising:
performing a standardized technical inspection process after the visual inspection for components having positive findings.

24. The method of claim 14, wherein the visual inspection is automatically performed.

25. The method of claim 14, further comprising:
performing a penetrant test or a surface protection method after the rinsing step f).

26. The method of claim 14, wherein a visual inspection for soft spots is performed prior to the step c) for alkaline pickling or prior to the step e) for acid pickling.
